(19) United States
(12) Patent Application Publication
(10) Pub. No.: US 2017/0319870 A1
(43) Pub. Date: Nov. 9, 2017

(54) PREVENTION AND SCREENING METHOD

(71) Applicants: Ann LIEBERT, Artarmon, New South Wales (AU); Roberta CHOW, Castle Hill, New South Wales (AU); Euahna VARIGOS, East Melbourne, Victoria (AU)

(72) Inventors: Ann LIEBERT, Artarmon, New South Wales (AU); Roberta CHOW, Castle Hill, New South Wales (AU); Euahna VARIGOS, East Melbourne, Victoria (AU)

(21) Appl. No.: 15/525,724

(22) PCT Filed: Nov. 12, 2015
(86) PCT No.: PCT/AU2015/000688

§ 371 Date: May 10, 2017

(30) Foreign Application Priority Data
Nov. 12, 2014 (AU) 2014904543

(51) Int. Cl. A61N 5/06; A61B 5/00

(52) U.S. Cl. CPC A61N 5/0613; A61B 5/4088; A61B 5/4082; A61N 2005/0659; A61N 2005/0662; A61N 2005/0651

(57) ABSTRACT

The present invention relates to a method of preventing or reducing one or more adverse post-anaesthetic effects in a patient, comprising treating the patient with low level laser therapy (LLLT) prior to anaesthesia and/or during anaesthesia and/or following anaesthesia, and to a method for assessing whether a patient is at risk of suffering from adverse post-anaesthetic effects, comprising determining whether the patient contains a polymorphism in a gene encoding one of the following proteins: (a) protein associated with assembly and disassembly of microtubules; (b)ion channel protein; (c) membrane stability protein; (d) Melanocortin systems proteins; (e) proteins associated with Alzheimer's and/or Parkinson's vulnerability; or (f) proteins associated with migraine.

### FIELD

The invention relates to a method of preventing adverse post-anaesthetic effects and to a method of screening for subjects at risk of suffering from adverse post-anaesthetic effects.

### BACKGROUND

General anaesthesia is a temporary state of unconsciousness which is induced in subjects before performing major surgery. The three main goals of anaesthesia are: lack of movement (paralysis), unconsciousness, and suppression of the stress response.

General anaesthesia is induced and maintained by administering various inhalation and/or intravenous drugs, known as general anaesthetics, which are specially formulated for general anaesthesia. Inhalation anaesthetics include desulfurane, enflurane, halothane, isoflurane, methoxyflurane, and sevoflurane. These anaesthetics are formulated to evaporate readily, and are administered by inhalation. Intravenous anaesthetics include barbituates such as: amobarbital, methohexital, thiamylal, and thiopental; benzodiazepines such as diazepam, lorazepam, midazolam; etomidate, ketamine and propofol.

Adverse effects experienced by some patients following general anaesthesia include nausea and vomiting, sore throat, pain, dizziness, confusion and cognitive impairment. While most adverse side effects of anaesthesia are temporary, some patients, particularly elderly patients, suffer from long term or even permanent cognitive impairment.

In view of the widespread use of general anaesthesia in surgery, what is needed is a means for screening for patients which are at risk of suffering from adverse post-anaesthetic effects, and a method of treating patients which are at risk of suffering from adverse post-anaesthetic effects, to reduce or prevent the onset of post-anaesthetic effects.

### SUMMARY

A first aspect provides a method of preventing or reducing one or more adverse post-anaesthetic effects in a patient, comprising treating the patient with low level laser therapy (LLLT) prior to anaesthesia and/or during anaesthesia and/or following anaesthesia.

An alternative first aspect provides a low level laser therapy for use in a method of preventing or reducing one or more adverse post-anaesthetic effects in a patient, wherein the patient is treated with low level laser therapy prior to anaesthesia and/or during anaesthesia and/or following anaesthesia.

A second aspect provides a method for assessing whether a patient is at risk of suffering from adverse post-anaesthetic effects, comprising determining whether the patient contains a polymorphism in a gene encoding one of the following proteins:

(a) protein associated with assembly and disassembly of microtubules;
(b) ion channel protein;
(c) membrane stability protein;
(d) melanocortin systems proteins;
(e) proteins associated with Alzheimer's and/or Parkinson's vulnerability;
(f) proteins associated with migraine,
wherein the patient is at risk of suffering from adverse post-anaesthetic effects when a polymorphism is detected.

A third aspect provides a method for assessing whether a patient is at risk of suffering from adverse post-anaesthetic effects, comprising determining whether the patient contains a polymorphism in a gene encoding one of the following proteins:

(a) protein associated with assembly and disassembly of microtubules;
(b) ion channel protein;
(c) membrane stability protein;
(d) melanocortin systems proteins;
(e) proteins associated with Alzheimer's and/or Parkinson's vulnerability;
(f) proteins associated with migraine,
wherein the patient is at risk of suffering from adverse post-anaesthetic effects when a polymorphism is detected; and treating the patient determined to be at risk of suffering from adverse post-anaesthetic effects with low level laser therapy prior to anaesthesia and/or during anaesthesia and/or following anaesthesia.

A fourth aspect provides a low level laser device or LED device when used for preventing or reducing adverse post-anaesthetic effects in a patient, wherein the device is arranged to administer low level laser therapy to the patient prior to anaesthesia and/or during anaesthesia and/or following anaesthesia.

A fifth aspect of the present invention provides a method of preventing or reducing post-anaesthetic dementia in a patient, comprising treating the patient with low level laser therapy (LLLT) prior to anaesthesia and/or during anaesthesia and/or following anaesthesia.

An alternative fifth aspect provides a low level laser therapy for use in a method of preventing or reducing post-anaesthetic dementia in a patient, wherein the patient is treated with low level laser therapy prior to anaesthesia and/or during anaesthesia and/or following anaesthesia.

A sixth aspect provides a method for assessing whether a patient is at risk of suffering from post-anaesthetic dementia, comprising determining whether the patient contains a polymorphism in a gene encoding one of the following proteins:

(a) protein associated with assembly and disassembly of microtubules;
(b) ion channel protein;
(c) membrane stability protein;
(d) Melanocortin systems proteins;
(e) proteins associated with Alzheimer's and/or Parkinson's vulnerability;
(f) proteins associated with migraine,
wherein the patient is at risk of suffering from post-anaesthetic dementia when a polymorphism is detected.

A seventh aspect provides a method for assessing whether a patient is at risk of suffering from post-anaesthetic dementia, comprising determining whether the patient contains a polymorphism in a gene encoding one of the following proteins:

(a) protein associated with assembly and disassembly of microtubules;
(b) ion channel protein;
(c) membrane stability protein;
(d) melanocortin systems proteins;
(e) proteins associated with Alzheimer's and/or Parkinson's vulnerability;
(f) proteins associated with migraine,
wherein the patient is at risk of suffering from post-anaesthetic dementia when a polymorphism is detected; and treating the patient determined to be at risk of suffering from post-anaesthetic dementia with low level laser therapy prior to anaesthesia and/or during anaesthesia and/or following anaesthesia.

An eighth aspect provides a low level laser device or LED device when used for preventing or reducing post-anaesthetic dementia in a patient, wherein the device is arranged to administer low level laser therapy to the patient prior to anaesthesia and/or during anaesthesia and/or following anaesthesia.

### BRIEF DESCRIPTION OF THE FIGURES

FIG. 1 is a photograph of a rat DRG following treatment with low level laser and staining with fluorescently labeled anti-□III tubulin and mitotracker red. Staining of small varicosities in the neurites are indicated by arrows.

### DETAILED DESCRIPTION

Varicosities are focal swellings in axons and dendrites of neurons which limit transmission of neural information. While formation of small varicosities in neurites is protective of neurons, formation of large varicosities has been associated with neuronal damage and death of the neuron. General anaesthesia has been shown to be associated with the appearance of large varicosities in neurites of neurons of patients. Without wishing to be bound by theory, the inventors believe that the formation of large varicosities in susceptible patients leads to onset of adverse post-anaesthetic effects such as post-anaesthetic dementia.

Without wishing to be bound by theory, the inventors believe that during anaesthesia, microtubules within neurons disassemble and reassemble, and that the varicosities in neurons caused by anaesthesia are due to reassembly of misfolded proteins associated with microtubules in susceptible individuals, and in particular, misfolding of prion protein.

As described herein, the inventors have found that administering low level laser therapy to neurons results in the formation of small varicosities with the accumulation of mitochondria within the small varicosities. The inventors reason that as small varicosities are protective of neurons, inducing small varicosities using low level laser therapy may be protective of neurons during anaesthesia in susceptible patients.

Without wishing to be bound by theory, the inventors believe that low level laser therapy reconfigures misfolded proteins to allow correct reassembly of microtubules. The inventors therefore believe that subjecting a patient who is susceptible to, for example, post-anaesthetic dementia to low level laser or LED prior to, during or after anaesthesia, causes a conformational shift in proteins, such as prion proteins, that may have misfolded, causing at least a portion of the misfolded proteins to return to the correct configuration. This reconfiguration of misfolded proteins triggers a global response to misfolded proteins, which results in a reduction in the formation of large varicosities, and promotes formation of small varicosities in the dendrite of neurons. The small varicosities are protective of the neuron and therefore prevent neuronal damage during anaesthesia. As a consequence, the patient does not suffer from the same neuronal damage or neuronal death, and is therefore less likely to suffer from adverse post-anaesthetic effects such as post-anaesthetic dementia, pain, nausea, vomiting, dizziness, blurred vision.

The invention therefore provides a method of preventing or reducing adverse post-anaesthetic effects in a patient, comprising treating the patient with low level laser therapy prior to and/or during and/or following anaesthesia.

As used herein, an “adverse post-anaesthetic effect” is an adverse condition that occurs following general anaesthesia. Examples of adverse post-anaesthetic effects include pain, nausea and vomiting, dizziness, blurred vision, dementia. In one embodiment, the adverse post-anaesthetic effect is post-anaesthetic dementia. As used herein, “post-anaesthetic dementia” is a decline in cognitive ability of a patient following general anaesthetic. Thus, the cognitive ability of the patient after anaesthetic will be reduced relative to the cognitive ability of the patient prior to anaesthesia. The decline in cognitive ability may be short term, long term, or in some cases, permanent. A decline in cognitive ability may include one or more of the following: confusion, lack of clarity, lack of rational thought processes, lack of memory, lack of awareness, lack of problem solving, lack of decision making, delirium, and/or lack of other mental processes. In one embodiment, the decline in cognitive ability is short term. A short term decline in cognitive ability is a decline in cognitive ability for a short period of time following anaesthesia. A short term decline in cognitive ability lasts no more than 14 days, and may last for from 1 hour 14 days, 1 hour to 13 days, 1 hour to 12 days, 1 hour to 11 days, 1 hour to 10 days, 1 hour to 9 days, 1 hour to 8 days, 1 hour to 7 days, 1 hour to 6 days, 1 hour to 5 days, 1 hour to 4 days, 1 to 72 hours, 1 to 48 hours, 1 to 36 hours, 1 to 24 hours, 1 to 18 hours, 1 to 12 hours, 1 to 8 hours, 1 to 6 hours, 1 to 4 hours, or 1 to 2 hours, following anaesthesia. In one embodiment, the decline in cognitive ability is long term. As used herein, a long term decline in cognitive ability is a decline in cognitive ability that last for greater than 14 days following anaesthesia. The long term decline in cognitive ability may, in some cases, last for greater than 3 months, 6 months, 12 months, 2 years, 3 years, or 5 years, following anaesthesia. In some embodiments, the long term decline in cognitive ability is a permanent decline in cognitive ability following anaesthesia. Post-anaesthetic dementia is sometimes referred to as postoperative cognitive dysfunction (POCD).

Methods for evaluating cognitive ability are known in the art and include, for example, the Mini Mental State Examination (MMSE), Abbreviated Mental Test (AMT), Six Item Screener (SIS), Clock Drawing Test (CDT), Mini-Cog, the General Practitioner Assessment of Cognition (GPCOG).

In one embodiment, those patients identified to be at risk of suffering from adverse post-anaesthetic effects, such as post-anaesthetic dementia, are administered LLLT to prevent or reduce the post-anaesthetic effect. However, it will be appreciated by those skilled in the art that the low level laser therapy could be administered to any patients undergoing anaesthesia, irrespective of whether they are at risk of developing adverse post-anaesthetic effects, as a precautionary measure.

As used herein, “preventing” means preventing a condition from occurring in a cell or patient that may be at risk of having or developing the condition, but does not necessarily mean that condition will not eventually develop, or that a subject will not eventually develop a condition. Preventing includes delaying the onset of a condition in a cell or subject. As used herein, “reducing” means reducing the extent or severity of a condition in a cell or patient relative to the extent or severity that would have been observed without applying the method. In one embodiment, preventing achieves the result of preventing the onset of adverse post-anaesthetic effects in a recipient patient.

As used herein, the term “patient” refers to a mammal such as a human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Typically the mammal is a human or primate. More typically, the mammal is a human. Although the present invention is exemplified in part using a rat model, this is not intended as a limitation on the application of the present invention to that species, and the invention may be applied to other species, in particular, humans.

Low level laser therapy refers to exposing a patient to photons from a low level laser or light-emitting diode (LED). As used herein, “low level laser” (LLL) is a laser that emits photons at a power density which is sufficient to have a biological effect but not sufficient to cause thermal damage to tissue. As used herein, a light-emitting diode is a semiconductor light source which emits photons at a power density which is sufficient to cause a biological effect but not sufficient to cause thermal damage to tissue. The biological effect is typically a photochemical reaction which is initiated in the cells treated with the photons. As used herein, “power density” is the amount of power delivered per unit area. Power density is typically expressed in Watts per square centimetre. The power density used in low level laser therapy is typically in the range of from 1 to 500 mW/cm², 1 to 100 mW/cm², 100 to 500 mW/cm², 200 to 400 mW/cm², 250 to 400 mW/cm², 250 to 350 mW/cm². The power density of a laser or LED may be adjusted by adjusting the power output of the laser or LED and/or the distance of the laser or LED from the patient.

As used herein, “energy density” is calculated by multiplying the power density by the amount of time (in seconds) the patient is subjected to the low level laser therapy. Energy density is expressed in Joules/cm². The energy density administered to the patient may be in the range of from 0.3-10 Joules/cm², 0.4-10 Joules/cm², 0.5-10 Joules/cm², 0.3-9 Joules/cm², 0.4-9 Joules/cm², 0.5-9 Joules/cm², 0.3-8 Joules/cm², 0.4-8 Joules/cm², 0.5-8 Joules/cm², 0.3-7 Joules/cm², 0.4-7 Joules/cm², 0.5-7 Joules/cm², 0.3-6 Joules/cm², 0.4-6 Joules/cm², 0.5-6 Joules/cm², 0.3-5 Joules/cm², 0.4-5 Joules/cm², 0.5-5 Joules/cm², 0.6-10 Joules/cm², 0.6-9 Joules/cm², 0.6-8 Joules/cm², 0.6-7 Joules/cm², 0.6-7 Joules/cm², 0.6-6 Joules/cm², 0.6-5 Joules/cm², 0.7-10 Joules/cm², 0.7-9 Joules/cm², 0.7-8 Joules/cm², 0.7-7 Joules/cm², 0.7-6 Joules/cm², 0.7-5 Joules/cm², 1-10 Joules/cm², 2-9 Joules/cm², 3-8 Joules/cm², 3-7 Joules/cm², 3-6 Joules/cm², 4-6 Joules/cm² or 4-5 Joules/cm², per dose.

The low level laser therapy (LLLT) is administered by subjecting the patient to photons emitted from a low level laser or LED. Typically, the LLLT is administered by subjecting a portion of the skin surface of the patient to photons. The photons are administered at a wavelength that promotes formation of small varicosities in neurons of the patient. The wavelength is in the range of from 300 nm to 1080 nm, 400 nm to 1050 nm, 400 nm to 1000 nm, 600 nm to 1000 nm, 450 to 950 nm, 450 nm to 910 nm, 500 nm to 950 nm, 600 nm to 950 nm, 700 to 950 nm, 800 to 950 nm, 850 to 950 nm, 880 nm to 930 nm, 500 nm to 900 nm, 600 nm to 900 nm, 600 to 800 nm, 600 to 700 nm, 700 nm to 900 nm, 800 nm to 900 nm, 810 nm to 850 nm, 820 nm to 840 nm, 825 nm to 835 nm, 650 nm to 665 nm, 650 nm to 660 nm. In one embodiment, the wavelength is 904 nm. In one embodiment, the wavelength is 658 nm.

In some embodiments, the photons are pulsed. The photons may be pulsed at a frequency in the range of 1-10,000 Hz, 1,000-10,000 Hz, 2,000-10,000 Hz, 3,000-10,000 Hz, 4,000-10,000 Hz, 4,000-9,000 Hz, 4,000-8,000 Hz, 4,000-7,000 Hz, 4,000-6,000 Hz, 5,000-10,000 Hz, 6,000-10,000 Hz, 7,000-10,000 Hz.

The patient is treated with LLLT prior to, during or following anaesthesia or combinations thereof

In one embodiment, the LLLT is administered prior to anaesthesia. The LLLT may be administered at a time within 4 weeks, 3 weeks, 2 weeks, 1 week, 96 hours, 72 hours, 48 hours, 24 hours, or immediately prior to anaesthesia. In one embodiment, the LLLT is administered 24 hours prior to administration of anaesthetic. In one embodiment, the LLLT is administered within 24 hours of administration of the anaesthetic, typically within 12, 11, 10, 9, 8, 7, 6, 5, 4, 3, 2 or 1 hour of administration of the anaesthetic.

Typically, the laser or LED is targeted to a site on the skin surface of the patient. The inventors envisage that the LLLT will exert its direct effect through absorption in cellular chromophores in neurons, as well as through an abscopal effect and accordingly, it is not necessary for the laser to be targeted to the neurons directly. As used herein, an “abscopal effect” is when a response to a stimulus is observed at a location distal to the site of administration of the stimulus. Without wishing to be bound by theory, the inventors believe that formation of small varicosities in the neurons of a patient in response to LLLT administered to a remote site, such as the skin surface, is through an abscopal effect in which photon absorption at the skin surface results in protein conformation changes, leading to cell to cell communication through a cascade of endogenous photon release, conformational changes in protein structure and signal transduction, resulting in reconfiguration of misfolded proteins in the neurons of the patient.

In various embodiments, the laser or LED is targeted to one or more of the following sites of the patient per treatment:

(a) Skin surface at the area of surgery;
(b) Skin surface near the spinal nerve root centrally above the spinous process (targeting dorsal root ganglia) and laterally 5 mm;
(c) Skin surface over the spinous process of C2 to sacral nerve roots encompassing the thoracic and lumbar spine, and laterally 5 mm;
(d) Skin surface over thoracic spine T3-T7;
(e) Skin surface over both temporal bones and forehead.

In one embodiment, there is provided a method of reducing or preventing adverse post-anaesthetic effects in a patient, comprising treating the patient with low level laser therapy (LLLT) prior to anaesthesia, wherein the LLLT is administered at a wavelength in the range of from 600 nm to 950 nm and an energy density per dose in the range of from 0.5 to 5 Joules/cm².

In one embodiment, there is provided a method of reducing or preventing one or more adverse post-anaesthetic effects selected from the group consisting of pain, nausea and vomiting, dizziness, blurred vision, dementia, the method comprising treating the patient with low level laser therapy (LLLT) prior to anaesthesia, wherein the LLLT is administered at a wavelength in the range of from 600 nm to 950 nm and an energy density per dose in the range of from 0.5 to 5 Joules/cm².

In one embodiment, there is provided a method of reducing or preventing post-anaesthetic dementia in a patient, comprising treating the patient with low level laser therapy (LLLT) prior to anaesthesia, wherein the LLLT is administered at a wavelength in the range of from 600 nm to 950 nm and an energy density per dose in the range of from 0.5 to 5 Joules/cm².

It will be understood that the specific dose level, energy density, wavelength and frequency of dosage for any particular patient may be varied and will depend upon a variety of factors including the age, body weight, general health, sex, diet, the anaesthetic administered, whether a combination of anaesthetics is administered, duration of the anaesthetic, extent of surgery and the susceptibility to postanaesthetic dementia of the patient undergoing therapy.

In some embodiments, the LLLT is administered to patients who are at risk of suffering from adverse post-anaesthetic effects, such as post-anaesthetic dementia.

In some embodiments, the patients will have a history of one or more adverse post-anaesthetic effects. Without wishing to be bound by theory, the inventors believe that the distribution of the severity of adverse post-anaesthetic effects in a patient population is bell-shaped, and that certain patients have a propensity to experience adverse post-anaesthetic effects.

The inventors believe that patients that are at risk of suffering from adverse post-anaesthetic effects, such as post-anaesthetic dementia, have a polymorphism in a gene encoding one of the following proteins:

(a) protein associated with assembly and disassembly of microtubules, such as for example, prion protein (PrPC), stathmin 1, Tau, A□ protein, synaptophysin protein;
(b) ion channel protein, such as for example, TREK, TRESK, calcium channel protein, VGCC, SOCC;
(c) membrane stability protein, such as for example, copper transport genes, MOA, serotonin receptor, NMDA, CREB1, dopamine receptors, adrenergic receptors, serotonin transporter CACNA1A, DREAM, protein encoded by gene for proopiomelanocortin prohormone (POMC) tyrosinase, Na⁺ transporters, K⁺ transporters, ATPase, BDNF, NGF, methylenetetrahydrofolate reductase (MLTHR);
(d) melanocortinin systems proteins, such as for example, proopiomelanocortin prohormone (POMC), melanocortin receptors MCR1, melanocortin receptor MCR3, agouti related protein, attractin, light response proteins (ADRAB);
(e) proteins associated with Alzheimer's and/or Parkinson's vulnerability, such as for example, parkin, ROBO1,2,3;
(f) proteins associated with migraine, such as, for example, proteins encoded by CACNA1, ATP1A2, SCNa1, TRESK genes.

Accordingly, the present invention provides a method for assessing whether a patient is at risk of suffering from an adverse post-anaesthetic effect, such as post-anaesthetic dementia, comprising determining whether the patient contains a polymorphism in a gene encoding one of the following proteins:

(a) protein associated with assembly and disassembly of microtubules, such as for example, prion protein (PrPC), stathmin 1, Tau, A□ protein, synaptophysin protein;

(b) ion channel protein, such as for example, TREK, TRESK, calcium channel protein, VGCC, SOCC;

(c) membrane stability protein, such as for example, copper transport genes, MOA, serotonin receptor, NMDA, CREB1, dopamine receptors, adrenergic receptors, serotonin transporter CACNA1A, DREAM, protein encoded by gene for proopiomelanocortin prohormone (POMC) tyrosinase, Na+ transporters, K+ transporters, ATPase, BDNF, NGF, methylenetetrahydrofolate reductase (MLTHR);

(d) melanocortin systems proteins, such as for example, proopiomelanocortin prohormone (POMC), melanocortin receptors MCR1, melanocortin receptor MCR3, agouti related protein, attractin, light response proteins (ADRAB);

(e) proteins associated with Alzheimer's and/or Parkinson's vulnerability, such as for example, parkin, ROBO1,2,3;

(f) proteins associated with migraine, such as for example, proteins encoded by CACNA1, ATP1A2, SCNa1, TRESK genes, wherein the patient is at risk of suffering from an adverse post-anaesthetic effect when a polymorphism is detected.

The present invention further provides a method for assessing whether a patient is at risk of suffering from an adverse post-anaesthetic effect, such as post-anaesthetic dementia, comprising determining whether the patient contains a polymorphism in a gene encoding one of the following proteins:

(a) protein associated with assembly and disassembly of microtubules, such as for example, prion protein (PrPC), stathmin 1, Tau, A□ protein, synaptophysin protein;

(b) ion channel protein, such as for example, TREK, TRESK, calcium channel protein, VGCC, SOCC;

(c) membrane stability protein, such as for example, copper transport genes, MOA, serotonin receptor, NMDA, CREB1, dopamine receptors, adrenergic receptors, serotonin transporter CACNA1A, DREAM, protein encoded by gene for proopiomelanocortin prohormone (POMC) tyrosinase, Na+ transporters, K+ transporters, ATPase, BDNF, NGF, methylenetetrahydrofolate reductase (MLTHR);

(d) melanocortin systems proteins, such as for example, proopiomelanocortin prohormone (POMC), melanocortin receptors MCR1, melanocortin receptor MCR3, agouti related protein, attractin, light response proteins (ADRAB);

(e) proteins associated with Alzheimer's and/or Parkinson's vulnerability, such as for example, parkin, ROBO1,2,3;

(f) proteins associated with migraine, such as for example, proteins encoded by CACNA1, ATP1A2, SCNa1, TRESK genes, wherein the patient is at risk of suffering from an adverse post-anaesthetic effect when a polymorphism is detected, and treating the patient determined to be at risk of suffering from adverse post-anaesthetic effects, such as post-anaesthetic dementia, with low level laser therapy prior to anaesthesia and/or during anaesthesia and/or following anaesthesia.

In one embodiment, present invention provides a method for assessing whether a patient is at risk of suffering from an adverse post-anaesthetic effect, such as post-anaesthetic dementia, comprising determining whether the patient contains a polymorphism in a gene encoding one of the following proteins:

(a) protein associated with assembly and disassembly of microtubules, such as for example, prion protein (PrPC), stathmin 1, Tau, A□ protein, synaptophysin protein;

(b) ion channel protein, such as for example, TREK, TRESK, calcium channel protein, VGCC, SOCC;

(c) membrane stability protein, such as for example, copper transport genes, MOA, serotonin receptor, NMDA, CREB1, dopamine receptors, adrenergic receptors, serotonin transporter CACNA1A, DREAM, protein encoded by gene for proopiomelanocortin prohormone (POMC) tyrosinase, Na+ transporters, K+ transporters, ATPase, BDNF, NGF, methylenetetrahydrofolate reductase (MLTHR);

(d) melanocortin systems proteins, such as for example, proopiomelanocortin prohormone (POMC), melanocortin receptors MCR1, melanocortin receptor MCR3, agouti related protein, attractin, light response proteins (ADRAB);

(e) proteins associated with Alzheimer's and/or Parkinson's vulnerability, such as for example, parkin, ROBO1,2,3;

(f) proteins associated with migraine, such as for example, proteins encoded by CACNA1, ATP1A2, SCNa1, TRESK genes, wherein the patient is at risk of suffering from an adverse post-anaesthetic effect when a polymorphism is detected, and treating the patient determined to be at risk of suffering from adverse post-anaesthetic effects, such as post-anaesthetic dementia, with low level laser therapy (LLLT) prior to anaesthesia, wherein the LLLT is administered at a wavelength in the range of from 600 nm to 950 nm and an energy density per dose in the range of from 0.5 to 5 Joules/cm².

In one embodiment, the protein associated with assembly and disassembly of microtubulesis is one or more proteins selected from the group consisting of prion protein (PrPC), strathmin 1, Tau, Aβ protein, and synaptophysin protein.

In one embodiment, the ion channel is one or more ion channels selected from the group consisting of TREK, TRESK, calcium channel protein, VGCC, and SOCC.

In one embodiment, the membrane stability protein is one or more proteins selected from the group consisting of copper transport genes, MOA, serotonin receptor, NMDA, CREB1, dopamine receptors, adrenergic receptors, serotonin transporter CACNA1A, DREAM, protein encoded by gene for proopiomelanocortin prohormone (POMC) tyrosinase, Na+ transporters, K+ transporters, ATPase, BDNF, NGF, and methylenetetrahydrofolate reductase (MLTHR).

In one embodiment, the melanocortin systems protein is one or more proteins selected from the group consisting of proopiomelanocortin prohormone (POMC), melanocortin receptors MCR1, melanocortin receptor MCR3, agouti related protein, attractin, and light response proteins (ADRAB).

In one embodiment, the proteins associated with Alzheimer's and/or Parkinson's vulnerability is one or more proteins selected from the group consisting of parkin, ROBO1,2,and 3.

In one embodiment, the proteins associated with migraine is one or more proteins selected from the group consisting of proteins encoded by CACNA1, ATP1A2, SCNa1 and TRESK genes.

A polymorphism may exist in a gene encoding one of the above proteins, or in a plurality of genes, of the patient. In one embodiment, the polymorphism is in one or more genes selected from the group consisting of a gene associated with cytoskeleton formation and modulation, such as the genes encoding TREK1, prion protein, POMC, αMSH, MC1R (the receptor for αMSH) or in genes encoding proteins such as TREK, calcium channel proteins, BDNF, agouti related protein, attractin, protein associated with neurotrophic signaling, protein associated with light response, or protein associated with Alzheimer's and/or Parkinson's vulnerability.

As used herein, a “polymorphism” is a difference in a gene of a patient when compared to the corresponding wild-type gene. As used herein, a “wild-type gene” is the gene of a patient that is not at risk of suffering from an adverse post-anaesthetic effect. The polymorphism may be determined by detecting the polymorphism in a sample of the patient. The sample may be, for example, blood, serum, plasma, tissue, cells, organs, bone marrow, cerebrospinal fluid, etc.

In one embodiment, the sample is a tissue. The tissue may be any sample from the patient, including blood, skin, buccal cells. Methods for obtaining samples from patients are known in the art.

The sample may be processed to enhance detectability of the polymorphism. The sample may be processed to enrich for nucleic acid such as DNA or mRNA. Methods for enrichment of DNA, RNA, including mRNA, are known in the art and are described in Sambrook, J., Russet D. W., ed. Molecular Cloning: A Laboratory Manual Volume 1, 2, 3. 2001. Cold Spring Harbor Laboratory Press.

Once a sample has been obtained from the subject, the sequence of the nucleic acid in the sample is compared with the corresponding sequence from a subject not at risk of suffering from an adverse post-anaesthetic effects to determine whether a polymorphism is present.

The polymorphism in a sample can be determined using methods known in the art, e.g., gel electrophoresis, capillary electrophoresis, size exclusion chromatography, sequencing, hybridization and/or arrays to detect the presence or absence of the polymorphism.

Methods of nucleic acid analysis to detect polymorphisms include, for example, microarray analysis, hybridization methods, sequencing analysis. Hybridisation methods include techniques such as Southern hybridisation analysis, Northern hybridisation analysis (see Current Protocols in Molecular Biology, Ausubel, F. et al., eds., John Wiley & Sons 2003; Sambrook, J., Russet D. W., ed. Molecular Cloning: A Laboratory Manual Volume 1, 2, 3. 2001. Cold Spring Harbor Laboratory Press). Sequencing methods include direct manual sequencing; and automated fluorescent sequencing. Other methods include, for example, single-stranded conformation polymorphism assays (SSCP); GC-clamped denaturing gel electrophoresis (CDGE); conformational sensitive gel electrophoresis (CSCE); denaturing gradient gel electrophoresis (DGGE), mobility shift analysis, restriction enzyme analysis; PCR; heteroduplex analysis; chemical mismatch cleavage (CMC); RNase protection assays.

In order to detect polymorphisms, a portion of genomic DNA encompassing the polymorphic site may be amplified using amplification techniques such as PCR. PCR methods are described in, for example, Sambrook, J., Russet D. W., ed. Molecular Cloning: A Laboratory Manual Volume 1, 2, 3. 2001. Cold Spring Harbor Laboratory Press. Guidelines for selecting primers for PCR amplification are well known in the art.

In one example, a sample (e.g., a sample comprising genomic DNA), is obtained from a subject. The DNA in the sample is then examined to determine polymorphisms in genes as described herein. The polymorphism can be determined by any method known in the art, including those described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In some embodiments, restriction digest analysis can be used to detect the existence of a polymorphism if the polymorphism results in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Ausubel et al., Current Protocols in Molecular Biology). The digestion pattern of the relevant DNA fragment indicates the presence or absence of a particular polymorphic variant of the polymorphism and is therefore indicative of the presence or absence of susceptibility to postanaesthetic dementia.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphism is determined.

Allele-specific oligonucleotides can also be used to detect the presence of a polymorphism e.g., through the use of dot-blot hybridization of amplified oligonucleotides with allele-specific oligonucleotide. An “allele-specific oligonucleotide” is typically an oligonucleotide of approximately 10-30 base pairs, that specifically hybridizes to a nucleic acid region that contains a polymorphism. An allele-specific oligonucleotide probe that is specific for particular a polymorphism can be prepared using standard methods (see Ausubel et al., Current Protocols in Molecular Biology). To determine if a patient comprises a polymorphism associated with an adverse post-anaesthetic effect, such as post-anaesthetic dementia, using a probe, in one form, a sample comprising DNA is obtained from the individual. PCR can be used to amplify DNA encompassing the polymorphic site. The amplified portion may be hybridised, using standard methods (see Ausubel et al., Current Protocols in Molecular Biology), with the oligonucleotide probe. The presence of specific hybridization of the probe to the DNA is then detected. Specific hybridization of an allele-specific oligonucleotide probe to DNA from the subject is indicative of susceptibility to an adverse post-anaesthetic effect.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence. Methods for the use of probes are known in the art and described in, for example, Sambrook, J., Russet D. W., ed. Molecular Cloning: A Laboratory Manual Volume 1, 2, 3. 2001. Cold Spring Harbor Laboratory Press; Ausubel et al., Current Protocols in Molecular Biology). In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. The probes may be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Polymorphisms may be detected using arrays comprising a nucleic acid probe that binds specifically to a sequence comprising a polymorphism in a gene of interest, and can be used to detect the absence or presence of said polymorphism, e.g., one or more SNPs, microsatellites, or minisatellites. The array may further include at least one area that includes a nucleic acid probe that can be used to specifically detect another marker associated with post-anaesthetic dementia.

In one form, the sequence of the marker from the patient to be tested is compared to a reference sequence. The reference sequence is typically the sequence of the same marker from a patient who is known to be not at risk of suffering from an adverse post-anaesthetic effect, such as post-anaesthetic dementia (wild-type). If the sequence of the patient being tested exhibits differences compared to the reference sequence, then the patient is at risk of suffering from an adverse post-anaesthetic effect, such as post-anaesthetic dementia.

The inventors envisage that patients may also be assessed for susceptibility to adverse post-anaesthetic effects, such as post-anaesthetic dementia, by assessing the patients previous experience with general anaesthetic, or by applying screening methods such as family history screening, and testing for neural membrane sensitivity and cytoskeleton sensitivity.

Family history screening may include screening for family history of adverse post-anaesthetic effects such as post-anaesthetic dementia, alzheimer's disease, migraine.

Without wishing to be bound by theory, the inventors believe that the formation of dysfunctional varicosities in neurons during anaesthesia is the result in part of disassembly of cytoskeleton proteins, influenced in part by prion proteins.

PrPC has a role in modulation of the cytoskeleton, through interactions with integrins, stathmins, and tubulins. Overexpression of PrPC results in disruption of microtubule architecture and the consequent shortening of intestinal villi and the homeostasis of epithelial renewal. Overexpression of PrPC (106-126) in serotonergic and noradrenergic neurons resulted in altered neurite extensions with contorted swellings that resembled varicosities. In neuropathic injury (in an animal model), there is a disruption of cytoskeleton structure in the dorsal root ganglion, with the formation of sympathetic varicosities, which is important as the mechanism behind neuropathic pain behaviours. This is a result of abnormal communication between sensory neurons and sympathetic fibres in the DRG. Therapeutic interventions using LLLT may be effective where microtubule disruption causes reversible varicosity formation,

Prion protein exists in at least two conformational states: first, the cellular α-helix-rich isoform (PrPC) and, second, the prion disease-associated β-sheet isoform (PrPSc). In humans, PrPC is a 32-kDa protein, with 253 amino acids encoded by the single-copy PRPN gene, located on chromosome 20. The protein has regions that are highly conserved in all vertebrates. When not in a disease state, the cellular prion protein has two isoforms with 208-209 amino acids: a membrane-bound form and a soluble cytosol (=secreted) form. The membrane-bound PrPC is a glycoprotein, attached by a glycosylphosphatidylinositol (GPI) anchor10 to lipid rafts on the outer leaflet of the cell membrane, as is the case with most GPI-anchored proteins. The soluble form is not glycosylated. Prion protein has an intrinsically disordered protein (IDP) component, in that the N-terminal end (amino acids 23-121) of the protein does not have a permanent tertiary structure but is flexible. The C-terminal end of the protein (amino acids 127-231) has a well-structured globular tertiary structure, folded into three α-helices and two short β-strands.

The structure of the disease form of prion protein is not fully known. It has the same primary structure (amino acid sequence) as PrPC, but the secondary structure has more β-sheet regions than the α-helix of PrPC and is able to form amyloid fibrils. The isoform of the disease is highly stable, resistant to proteolytic enzymes, and self-replicating. The presence of PrPC is necessary for progression to prion disease and for neurotoxicity to occur, but the actual mechanism that controls the misfolding of PrPC is not known.

The PRPN gene is expressed in many cells of the body, but is most highly expressed in cells in the nervous system. Likewise, the protein PrPC is ubiquitous in cells of the body, but is found most abundantly in nervous system cells; in neurons (cell body and synaptic membrane) of the hippocampus, cortex, thalamus, cerebellum, and medulla; and in glial cells, including astrocytes. In most cells, PrPC is almost entirely membrane bound, with very little found in the cytoplasm. In some cells, however, such as neurons in the hippocampus, thalamus, and neocortex, the cytosol form of PrPC is commonly found. Both membrane-bound and soluble forms of PrPC are found in the cerebral spinal fluid. Membrane-bound PrPC can be secreted from cells into the extracellular matrix (ECM) in exosomes.

PrPC has been shown to have a role in the regulation of the neuroendocrine secretion of the pituitary molecule proopiomelanocortin prohormone (POMC) in an animal model. POMC is also regulated by p53, which is a target of PrPC. Oversecretion of PrPC over long periods resulted in destruction of POMC secretory granules by crinography (lysosome mediated). POMC is a precursor molecule in the melanocortin system, and specifically, a precursor molecule in the formation of melanin and hormones ACTH, αMSH (an inhibitor of NF-κB), β-opioid, and thyroid; and is therefore involved in energy homeostasis, autonomic regulation, pain regulation, and the pain and anaesthetic response of red-headed women with MCR1 receptor poymorphims. In this regard, people with polymorphisms in their melanocortin system are a group of patients that may be at risk of suffering from post-anaesthetic dementia. Importantly, αMSH is an inhibitor of NF-κB, which may also be upregulated by PrPC via ROS signaling. Without wishing to be bound by theory, the inventors believe that the link between PrPC, αMSH, and NF-κB suggests that PrPC plays a part in the anaesthetic response of elderly patients who suffer post-anaesthetic dementia, possibly involving the role of PrPC in cytoskeleton organization.

Another possibility envisaged by the inventors in relation to the interaction between PrPC and the melanocortin system is the close proximity on chromosome 20 (in humans) of the PrPC gene to critical pigmentation genes, including genes for agouti signaling protein (ASIP), attractin (ATRN), and melanocortin 3 (MC3) neural anti-inflammatory receptor. This proximity may link pigmentation to regulation of PrPC and to an interrelationship between PrPC gene expression and PrPC-regulated disease, especially given the effect of the temporary disruption of the cytoskeleton during general anesthesia and that PrPC interacts with MAPs and has a role in microtubule assembly and disassembly.

In addition to the two mechanisms for anaesthesia vulnerability discussed above, it has been demonstrated that there is a link between protein 14-3-3, calcineurin, and PAR-1/MARK pathway, which results in the coupling of microtubule dynamics and neuronal excitability through TRESK channels. TRESK is the ion channel most sensitive to anaesthetics such as halothane and isoflurane, mediating the suppression of wakefulness, awareness, and memory. PrPC has also been linked to protein 14-3-3100 and calcineurin giving a further link (via TRESK channels) to cytoskeleton dynamics. Thus, without wishing to be bound by theory, the inventors believe that PrPC has a role in postanaesthetic disease vulnerability. Implications for treatment include the screening of patients undergoing anaesthesia for TRESK polymorphisms and polymorphisms in any of the other genes described herein, and the targeting of PrPC for neuroprotection in relation to postanaesthetic dementia.

The inventors believe that vulnerability to postanaesthetic dementia and other adverse post-anaesthetic effects is mediated through disassembly of microtubule proteins through an interaction with prior protein. The inventors believe that this disassembly can be corrected in part through subjecting the patient to low level laser therapy.

In order to exemplify the nature of the present invention such that it may be more clearly understood, the following non-limiting examples are provided.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Example 1

### Treatment of Rat DRG Neurons with Low Level Laser

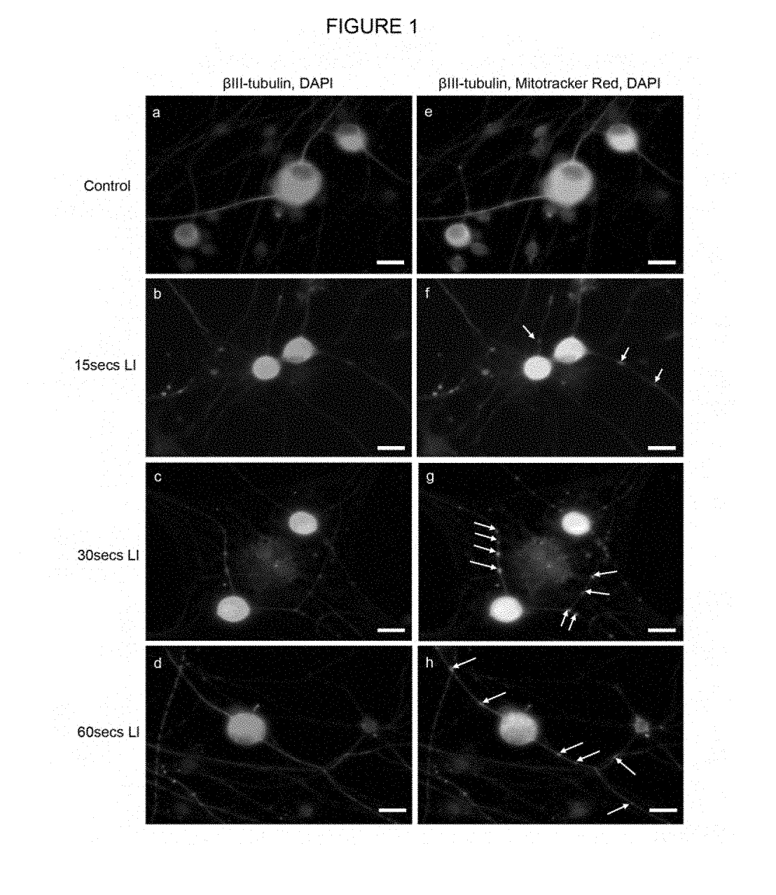

To assess the effects of low level laser on neurons, rat dorsal root ganglion (DRG) neurons were treated with 830 nm low level laser for 0 seconds, 15 seconds, 30 seconds and 60 seconds in the presence of anti-βIII-tubulin and mitotracker red. The results are shown in FIG. 1. In FIG. 1, small mitotracker red staining varicosities in dendrites of the neurons are shown with a white arrow.

The results indicate that treatment of DRG neurons with low level laser resulted in formation of small varicosities containing mitochondria in the dendrites of the neuron.

Thus, treatment of neurons induces the formation of small varicosities. The inventors therefore believe that, as small varicosities are protective of neurons, the administration of low level laser prior to or during anaesthesia will stimulate the production of small varicosities, resulting in a protective response in the neuron during anaesthesia.

### Example 2

### Low Level Laser Therapy

An example of low level laser therapy protocol suitable for treating a patient undergoing general anaesthetic is as follows:

Using a Low Level Laser (available from, for example, Irradia), the following treatment is carried out as early as several months prior to surgery, with a treatment made four weeks prior to surgery, once a week up until the surgery and in some cases, 2 treatments per week for 4 weeks), 24 hours prior to surgery (minimum dose), and on the day of surgery (such as 1 hour before anaesthesia):

1. Laser 658 nm, 808 nm or 904 nm at an appropriate dose window, such as 4-5 joules/cm², applied to area of surgery;

2. Laser 658 nm, 808 nm or 904 nm at an appropriate dose window, such as 4-5 joules/cm², applied to point over the corresponding spinal nerve root centrally above the spinous process (targeting dorsal root ganglia) and laterally 5 mm (3 pts);

3. Laser 658 nm, 808 nm or 904 nm at an appropriate dose window, such as 4-5 joules/cm², applied over the spinous process of C2 to to sacral nerve roots encompassing the thoracic and lumbar spine, and laterally 5 mm (3 pts);

4. Laser 658 nm, 808 nm or 904 nm at an appropriate dose window, such as 4-5 joules/cm², applied over thoracic spine T3-T7 4-5 joules per point for thoracic sympathetic chain (6 pts);

5. Laser 658 nm, 808 nm or 904 nm at an appropriate dose window, such as 4-5 joules/cm², applied to both temporal bones and forehead.

A further example of low level laser therapy protocol suitable for treating a patient undergoing general anaesthetic is as follows:

Using a Low Level Laser (available from, for example, Irradia), the following treatment is carried out as early as several months prior to surgery, with a treatment made four weeks prior to surgery, once a week up until the surgery and in some cases, 2 treatments per week for 4 weeks), 24 hours prior to surgery (minimum dose), and on the day of surgery (such as 1 hour before anaesthesia):

1. Near infrared laser 810 nm and LED laser 660 nm or 850 nm at an appropriate dose window, such as between 0.5-3.5 joules/cm², applied to various distal sites, to spinous processes and corresponding segmental dermatomes relevant to the surgical site and to the local site of operation.

2. LED laser 660 nm or 850 nm at an appropriate dose window, such as between 0.5-3.5 joules/cm², applied to various distal sites, to spinous processes and corresponding segmental dermatomes relevant to the surgical site and to the local site of operation.

### Example 3

### Treatment of Patient Preoperatively with LLLT

A 23 year old female patient who had previously experienced nausea, vomiting and significant pain following general anaesthesia and surgery was treated with LLLT prior to anaesthesia for a hysterectomy.

Prior to undergoing the hysterectomy, the patient was treated with a 658 nm wavelength laser (available from, for example, Silberbauer, Austria) delivered at a dose of either 0.5 or 2-5 joules/cm², at selected areas distal to the surgical site, and locally at the proposed surgical site and at relevant segmental dermatomes, each treatment was adjusted according to clinical assessment. Additional modified treatments up to several months prior to surgery in this case for pain management were necessary and then four weeks, three weeks two weeks and one week prior to the immediate preoperative treatment within 24 hours of the anaesthetic being administered.

Following surgery, the patient experienced no post-anaesthetic nausea and vomiting, was relaxed, and was far less emotional than she had anticipated considering the significance of the operation. She stated she felt “clear headed” compared to previous surgery. She was also surprised at how moderate the pain was, and the patient required minimal administration of narcotic analgesics. (These observations were commented and noted by nursing staff and parents).

### Example 4

### Treatment of Patient Preoperatively with LLLT

A 43 year old patient was treated with LLLT prior to surgery for cervical fusion cage and disc repair under a general anaesthetic.

The patient was treated 4 days and 1 day prior to the operation with 658 nm wavelength laser delivered at a dose of either 0.5 or 2-5 joules/cm² to areas of significance away from the surgical site, and with 658 nm wavelength laser delivered at a dose of either 0.5 or 2-5 joules/cm² and with 810 nm laser (available from, for example, Thor Photomedicine Ltd. UK) delivered at a dose of 2-5 joules/cm² locally to the surgical area, and to the relevant segmental dermatomes. In addition, the patient was treated with 660 nm LED for periods of up to 1 minute to local cervical areas, segmental dermatomes and peripheral areas.

Following surgery, the patient noted a postoperative clarity that was not present in 5 previous less major surgical procedures with general anaesthetic.

The patient experienced no pain for 24 hours post-anaesthesia, and tolerated pain during the hospital stay with minimal analgesia. (This was observed and noted by treating nursing staff)

### Example 5

### Treatment of Patient Preoperatively with LLLT

A 48 year old patient was treated with LLLT prior to knee surgery. The patient had 7 previous surgeries with general anaesthetic without LLLT, in which she consistently suffered serious pain and poor recovery.

The patient was treated 1 day prior to the operation with 658 nm wavelength laser with a dose of either 0.5 or 2-5 joules/cm² at peripheral and distal areas to the surgical site, at the local surgical site and at the relevant segmental dermatomes.

Following surgery, the patient noted a marked postoperative clarity that was not present in the 7 previous similar surgical procedures with general anaesthetic, each with the same surgeon and anaesthetist.

The patient also found that they were not overwhelmed by the operation or by pain, and found pain control easier to manage with reduced drug requirement and early discharge interstate.

### Example 6

### Effect of LLLT on Post-Anaesthetic Recovery

Following treatment of patients with LLLT prior to administration of general anaesthetic, the following effects were generally observed:

1. Patients appear to have improved postoperative clarity and are often very alert and reading when back in the ward following their operation;

2. Patients comment that they feel better than they expected or better than with previous similar surgery and anaesthesia;

3. Patients have rational thought processes;

4. With rational thought processes, patients have less catastrophizing;

5. Patients do not become overwhelmed with the pain associated with major surgery;

6. Patents have reduced pain scores and reduced requirement for narcotic and strong analgesics immediately following surgery and subsequent reduction in side effects of these drugs such as nausea and vomiting, confusion and hallucinations;

7. Patients have an ability to detach from what is often experienced as an overwhelming experience after major surgery. It is believed that the ability to detach from the overwhelming experience following surgery and general anaesthesia is in part due to an improved cognition of the patient. It is a rational thought process and a clarity of the situation with an objective analysis rather than being immersed in the overwhelming experience of pain and surgery post operatively.

The above indicate that LLLT administered prior to general anaesthetic reduces or prevents cognitive decline and other adverse post-anaesthetic effects.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method of preventing or reducing one or more adverse post-anaesthetic effects in a patient, comprising treating the patient with low level laser therapy (LLLT) prior to anaesthesia and/or during anaesthesia and/or following anaesthesia.

2. The method of claim 1, wherein the adverse post-anaesthetic effect is post-anaesthetic dementia.

3. The method of claim 2, wherein the post-anaesthetic dementia is long term.

4. The method of claim 1, wherein the LLLT is administered at a wavelength in the range of from 300 nm to 1000 nm.

5. The method of claim 4, wherein the wavelength is in the range of from 600 to 1000 nm.

6. The method of claim 1, wherein the LLLT is administered at an energy density in the range of from 0.5 to 5 Joules/cm².

7. The method of claim 1, wherein the LLLT is administered prior to anaesthesia.

8. A method of reducing or preventing adverse post-anaesthetic effects in a patient, comprising treating the patient with low level laser therapy (LLLT) prior to anaesthesia, wherein the LLLT is administered at a wavelength in the range of from 600 nm to 950 nm, and an energy density per dose in the range of from 0.5 to 5 Joules/cm².

9. A method of reducing or preventing one or more adverse post-anaesthetic effects selected from the group consisting of pain, nausea and vomiting, dizziness, blurred vision, dementia, the method comprising treating the patient with low level laser therapy (LLLT) prior to anaesthesia, wherein the LLLT is administered at a wavelength in the range of from 600 nm to 950 nm and an energy density per dose in the range of from 0.5 to 5 Joules/cm².

10. A method of reducing or preventing post-anaesthetic dementia in a patient, comprising treating the patient with low level laser therapy (LLLT) prior to anaesthesia, wherein the LLLT is administered at a wavelength in the range of from 600 nm to 950 nm and an energy density per dose in the range of from 0.5 to 5 Joules/cm².

11. (canceled)

12. (canceled)

13. (canceled)

14. (canceled)

15. (canceled)

16. (canceled)

17. (canceled)

18. A low level laser device or LED device when used for preventing or reducing post-anaesthetic dementia in a patient undergoing anaesthesia, wherein the device is arranged to administer LLLT to the patient prior to anaesthesia, and/or during anaesthesia and/or following anaesthesia.
